(19) United States
(12) Patent Application Publication
(10) Pub. No.: US 2024/0002942 A1
(43) Pub. Date: Jan. 4, 2024

(54) METHOD FOR PREIMPLANTATION GENETIC SCREENING OF EMBRYOS

(71) Applicant: REPROBIOGEN INC., Toronto (CA)

(72) Inventors: Clifford L. LIBRACH, Toronto (CA); Svetlana MADJUNKOVA, Toronto (CA); Valeriy KUZNYETSOV, Toronto (CA)

(21) Appl. No.: 18/255,113

(22) PCT Filed: Apr. 28, 2021
(86) PCT No.: PCT/CA2021/050586

§ 371 Date: May 31, 2023

Related U.S. Application Data
(60) Provisional application No. 63/121,463, filed on Dec. 4, 2020.

(51) Int. Cl. C12Q 1/6883; C12N 5/073; C12Q 1/6869

(52) U.S. Cl. CPC C12Q 1/6883; C12N 5/0604; C12Q 1/6869; C12Q 2600/156; C12Q 2600/16

(57) ABSTRACT

A non-invasive method for genetic screening prior to embryo implantation is described. Fertilized oocytes are cultured on day 1 followed by removal of residual cumulus/corona cells, reducing maternal contamination; isolating oocytes and culturing individually from days 1-4 with serum protein supplement; conducting laser zona breaching on day 4 allowing embryonic cell free DNA (cfDNA) into the culture medium; washing oocytes with fresh medium to produce a medium containing day 4 cfDNA; transferring into fresh medium under oil, and culturing until day 5, 6, or 7 to form an expanded blastocyst which is transferred on day 5/6/7 into a drop of medium; exposing the expanded blastocyst to laser pulse to extrude blastocoel fluid containing embryonic cfDNA, obtaining day 5/6/7 cfDNA; and conducting genetic screening of cfDNA using whole genome amplification (WGA). The method may be used for genetic screening of embryos for aneuploidy by testing WGA for whole chromosome copy number.

### CROSS REFERENCE TO RELATED APPLICATIONS

This application claims the benefit of and priority to U.S. Provisional Patent Application No. 63/121,463, filed on Dec. 4, 2020, the content of which is hereby incorporated by reference.

### FIELD

The present disclosure relates generally to a non-invasive method for genetic screening prior to implantation of an embryo, such as for use in determining aneuploidy.

### BACKGROUND

Genetic screening for aneuploidy is desirable prior to embryo implantation in the context of assisted reproductive technologies (ART). Aneuploidy exists when an abnormal number of chromosomes are present in a cell, such as a cell having 45 or 47 chromosomes instead of the normal number, ie. 46. Preimplantation Genetic Testing for Aneuploidies (PGT-A) is performed in the context of embryo selection to increase the chances of success of in vitro fertilization (IVF) technologies. Minimally invasive or non-invasive methods for preimplantation genetic testing (NIPGT) are desirable, to minimize the risk to the embryo that may occur due to invasive sampling methods.

Preimplantation genetic testing without trophectoderm (TE) biopsy is an attractive approach to avoid any potential risk of an invasive procedure. PGT-A can categorize embryos by chromosomal profile, allowing a cohort of embryos to be assessed for highest likelihood of a positive outcome.

It is desirable to innovate and optimize non-invasive methods for preimplantation genetic testing to increase accuracy of information, as well as the chances of success in reproductive technologies.

### SUMMARY

It has been found that that collection of both spent blastocyst culture media and blastocoels fluid together as one non-invasive sample can increase the quantity and quality of cell-free embryonic nuclear DNA (cfeDNA) compared with spent embryo culture media alone or blastocoel fluid alone.

There is described herein a non-invasive method for genetic screening prior to implantation of an embryo, said method comprising: culturing fertilized oocytes in a culture medium on day 1 of fertilization; removal of residual cumulus/corona cells from culture medium by pipetting and washing with fresh medium on culture day 1 to reduce maternal contamination; isolating fertilized oocytes and culturing individually from day 1 to day 4 in a culture medium comprising a serum protein supplement; conducting laser zona breaching on day 4 to allow embryonic cell free DNA (cfDNA) into said culture medium, and subsequently washing the fertilized oocytes with fresh medium to produce a medium containing day 4 cfDNA; transferring said washed fertilized oocytes on day 4 into fresh culture medium under oil, and culturing until day 5, day 6, or day 7 to thereby form an expanded blastocyst; transferring said expanded blastocyst on day 5, day 6, or day 7 (herein “day 5/6/7”) into a fresh drop of culture medium; exposing said expanded blastocyst to a laser pulse to extrude blastocoel fluid containing embryonic cell free DNA (cfDNA) in the fresh drop of culture medium to obtain day 5/6/7 cfDNA; and conducting genetic screening of the cfDNA using whole genome amplification (WGA) prior to implantation of the embryo.

The method may be used for conducting genetic screening for aneuploidy using WGA to determine whole chromosome copy number (WCN) as an indicator of aneuploidy.

The described method involves preparation of oocytes for fertilization, culturing the embryos from day 4-day 5/6/7 and collection of the spent media and blastocoel fluid for analysis as a one-step procedure, which advantageously permits reliable and non-invasive preimplantation genetic screening for aneuploidy (PGT-A). Certain aspects of the described method represent improvements over previous NIPGT methods, and may have certain advantages. For example, by including an extra washing step on day 1 to remove residual cumulus/corona cell contamination by pipetting and washing with fresh medium, superior results may be obtained.

Conducting laser zona breaching on day 4 allows extrusion of embryonic cfDNA into culture media. The culturing of fertilized oocytes from day 4 to day 5/6/7 can improve outcomes. Further, WGA cell free DNA being enzymatically treated before being used for library preparation has the advantage of improved results. Exo nuclease I treatment combined with SAP (Exo-SAP-I) to remove single stranded DNA renders improved outcomes.

Other aspects and features of the present disclosure will become apparent to those ordinarily skilled in the art upon review of the following description of specific embodiments in conjunction with the accompanying figures.

### BRIEF DESCRIPTION OF THE FIGURES

Embodiments of the present disclosure will now be described, by way of example only, with reference to the Figures.

FIG. 1 depicts TE (A, B & C) versus ICM (A, B, and C), showing an increasing apoptosis gradient. Modified SART grading was conducted according to Heitmann et al. (2013). 102 fully expanded blast were utilized from 28 subjects 29-40 years of age (average age: 35.0±3.3). Good quality >BB: 1/2AA, AB, BA, BB, N=55; Average/low quality <BB: 1/2AC, CA, BC, CB, CC, (N=47).

FIG. 2 depicts a copy number variation (CNV) plot showing copy number per chromosomal position for Embryo I (TE: 47, XY, +22, upper panel; miPGT: 47, XY, +22, lower panel).

FIG. 3 depicts a CNV plot showing copy number per chromosomal position for Embryo II (TE: 47, XX, +16 upper panel; miPGT: 47, XX, +16, lower panel).

FIG. 4 depicts a CNV plot showing copy number per chromosomal position for Embryo III (TE: 45, XX, −11, upper panel; miPGT: 45, XX, −11, lower panel).

FIG. 5 depicts a CNV plot showing copy number per chromosomal position for Embryo IV (TE: 46, XY, mosaic loss: −4q22.1 −4qter, 100 Mb, 20%, upper panel; miPGT: 46, XY, mosaic gain: +4q22.1 −4qter, 100 Mb, 30%, lower panel).

FIG. 6 depicts a copy number per chromosomal position for Embryo a (TE: 45, XY, −16, upper panel; miPGT-1: 45, XY, −16, middle panel; miPGT-2: 45, XY, −16, lower panel).

FIG. 7 depicts a copy number per chromosomal position for Embryo b (TE: 47, XX, +22, upper panel; miPGT-1: 47, XX, +22, middle panel; miPGT-2: 47, XX, +22, lower panel).

### DETAILED DESCRIPTION

The non-invasive method for genetic screening described herein permits screening prior to embryo implantation for such reproductive technologies as in vitro fertilization. Fertilized oocytes are cultured on day 1 and residual cumulus/corona cells are removed to reduce maternal contamination. Fertilized oocytes are then isolated and cultured individually from days 1-4 with a serum protein supplement. Laser zona breaching is conducted on day 4, allowing embryonic cell free DNA (cfDNA) into the culture medium. Fertilized oocytes are then washed with fresh medium to produce a medium containing day 4 cfDNA, and transferred into fresh medium under oil, and cultured until day 5/6/7 to form an expanded blastocyst. The expanded blastocyst is then transferred on day 5/6/7 into a drop of medium which is then exposed to a laser pulse to extrude blastocoel fluid containing embryonic cfDNA, thus obtaining day 5/6/7 cfDNA. Genetic screening of cfDNA is then conducted using whole genome amplification (WGA).

The step of conducting genetic screening can comprise aneuploidy testing using WGA to determine whole chromosome copy number (WCN) as an indicator of aneuploidy.

The removal of residual cumulus/corona cells from culture medium by pipetting and washing may comprise at least three washes with fresh culture medium, optionally followed by microscopic inspection.

The culture medium for the individually cultured fertilized oocytes from day 1 to day 4 can be, for example Sage 1-Step medium, utilized under oil in a culture medium droplet of about 25 μL.

The washing step that occurs after the laser zona breaching on day 4 may comprise three washings to remove residual cumulus/corona cells. Following the three washings, the fertilized oocyte can then be transferred to fresh culture medium comprising Global HP medium with human serum albumen (HAS), under oil, in a culture medium droplet of about 15 μl.

The expanded blastocyst, on day 5, day 6, or day 7 (herein referenced interchangeably as “day 5/6/7”) comprises a visible inner cell muss prior to said laser pulse.

The genetic screening of the cfDNA can be assessed in the spent culture media, in the blastocoel fluid, or in both. The cfDNA can be enzymatically treated with Exo nuclease I and Shrimp Alkaline phosphatase (Exo-SAP-IT) to remove single stranded DNA prior to whole genome amplification (WGA).

Whole genome amplification (WGA) can be conducted using, for example: a SurePlex™ kit, quantified with a Qubit 3.0™ fluorimeter, and next-generation sequencing (NGS) is conducted with VeriSeq™ PGS. Equivalent methodologies may be employed. The whole genome amplification (WGA) conducted with the SurePlex™ kit may involve 14 pre-amplification cycles for preparation of a library of sequences.

The DNA resulting from WGA can be subjected to PCR amplification followed by: Sanger sequencing, Single base extension analysis, or short tandem repeat (STR) analysis. Fluorescent markers may be used for short tandem repeat (STR) analysis.

The step of conducting genetic screening of the cfDNA using whole genome amplification (WGA) may employ the preparation of a cfDNA library using NexteraXT™ dual index set A-D with 16 amplification cycles, for example.

The step of conducting genetic screening may comprise copy number variation (CNV) analysis conducted with NxClinical™ software against a reference set from cell free embryonic DNA from euploid embryos.

Following the genetic screening method described herein, the embryo can be implanted into a human subject if it satisfies a requirement of the genetic screening, for example, if aneuploidy is not indicated in the genetic screening. Embryos that satisfy the requirement of the genetic screening may be frozen prior to implantation.

To gain a better understanding of the invention described herein, the following examples are set forth. It should be understood that these examples are for illustrative purposes only. Therefore, they should not limit the scope of this invention in any way.

### EXAMPLE 1

### Non-Invasive Cell-Free Human Embryo Aneuploidy Testing (Nipgt-A) Utilizing Combined Spent Embryo Culture Medium and Blastocoel Fluid

Abstract: In this example, a non-invasive method for cell-free human embryo aneuploidy testing (nipgt-a) is conducted utilizing combined spent embryo culture medium and blastocoel fluid. The method may be utilized as a clinical assay for rapid pre-screening. Preimplantation genetic testing for aneuploidies (PGT-A) using trophectoderm (TE) biopsy samples is labour intensive, invasive, skill dependant, and subject to sampling bias. In this example, the efficacy and factors affecting accuracy of the method of non-invasive preimplantation genetic testing for aneuploidy (NIPGT-A) are evaluated. The method uses cell-free embryonic DNA (cfeDNA) in spent embryo culture medium (SEM) combined with blastocoel fluid (BF) to increase the amount of assayable cfeDNA. NIPGT-A results (n=145 embryos) were compared with standard PGT-A analysis of the corresponding trophectoderm biopsy, and it was found that accuracy of NIPGT was not related to blastocyst morphological grade. Importantly, it is herein established that for cfeDNA analysis, the SurePlex™ whole genome amplification (WGA) kit can be utilized without an additional cell lysis/extraction DNA step. This efficiency can reduce the risk of maternal contamination. Regarding origin of embryonic cfeDNA, the average amount of NIPGT-A WGA-DNA obtained from blastocysts, as well as the size of NIPGT-A WGA-DNA fragments, indicates it is unlikely that apoptosis is the primary mechanism of DNA release from the inner cell mass (ICM) and TE into BF and SEM.

### Introduction & Background

Preimplantation genetic testing for aneuploidies (PGT-A) using trophectoderm (TE) biopsy and next-generation sequencing (NGS) as a testing platform for embryo selection has significantly improved ongoing pregnancy rates per transfer, shortened the time to pregnancy (through avoidance of transferring aneuploid embryos destined to fail), reduced multiple pregnancies by transferring single euploid embryos, reduced miscarriage rates and reduced risk of aneuploid pregnancies (Dandouh et al., 2015; Munne, 2018; Friedenthal et al., 2018; Rubio C, Rodrigo L, et al., 2019). However, there are three main challenges of the preimplantation genetic testing associated with trophectoderm biopsy samples: firstly, TE biopsy is labour intensive (Capalbo et al., 2018; Fang et al., 2019); secondly, TE biopsy is invasive and skill dependant (Guzman et al., 2019; Zhang S. et al., 2016); and thirdly it is subject to sampling bias, and thus the TE biopsy may not accurately represent the inner cell mass (ICM) and remainder of the TE (Maxewll et al, 2016; Popovic et al., 2018; Victor et al., 2019). Furthermore, although there has been no reported increase in the risk of adverse perinatal outcomes, such as pre-term birth and low birth weight, following invasive PGT compared with IVF without embryo biopsy (Sunkara et al., 2017), conclusive evidence regarding the long-term health of the offspring after embryo biopsy will take some time to obtain (Capalbo et al., 2018; He et al., 2019).

Cell-free embryonic nuclear DNA (cfeDNA) has been found in both blastocoel fluid (Palini et al., 2013; Tobler et al., 2015; Zhang Y. et al., 2016) and spent embryo culture medium (Galluzzi et al., 2015; Wu et al., 2015; Stigliani et al., 2013). Non-invasive preimplantation genetic testing (NIPGT) using the cfeDNA of spent embryo culture medium (SEM) and/or blastocoel fluid (BF) has the potential to eliminate the need for embryo biopsy, thereby avoiding potential risks related to that invasive procedure (Neal et al., 2017; Shamonki et al., 2016). Moreover, NIPGT is less labour intensive and potentially more cost-effective method. In addition, NIPGT-A, which is based on sequencing of cfeDNA released from both TE and ICM cells (Huang et al., 2019; Kuznyetsov et al., 2018), may better represent the entire embryo compared to TE biopsy alone (Ben-Nagi et al., Handyside, 2016).

The following research approaches are known for collecting cell-free embryonic DNA for non-invasive aneuploidy testing (NIPGT-A):
1) Blastocoel fluid aspiration using an ICSI pipette (Capalbo et al, 2018; Tobler et al., 2015; Magli et al., 2016).
2) Spent embryo culture medium collection (Rubio C, Rodrigo L, et al., 2019; Shamonki et al., 2016; Ho et al., 2018; Jiao et al., 2019; Vera-Rodriguez et al., 2018).
3) Combined spent embryo culture medium and blastocoel fluid (SEM+BF) collected without using an ICSI pipette (Kuznyetsov et al., 2018; Jiao et al., 2019; Li et al., 2018).

Attempts to use cfeDNA for non-invasive preimplantation aneuploidy testing brings to light several factors that could potentially affect the accuracy of this approach. These include maternal contamination by cumulus and corona cells (Vera-Rodriguez et al, 2018), cfeDNA degradation, low amounts of cfeDNA, variable DNA amplification efficacy and yield (Belandres et al., 2019; Poli et al., 2019), short DNA fragments (Zhang Y, et al. 2016), and reduced concordance rate with TE biopsy results.

It has been shown that cfeDNA testing using spent embryo culture medium on days 5 or 6 has the potential to detect chromosomal aneuploidy (Shamonki et al., 2016; Kuznyetsov et al., 2018; Ho et al., 2018; Vera-Rodriguez et al., 2018; Li et al., 2018; Xu et al., 2016). It has been hypothesized that cfeDNA is correlated with apoptotic events (Ben-Nagi et al., 2019; Hammond et al., 2016). If this is true, it should follow that lower quality blastocysts, which generally have higher degrees of apoptosis, would result in higher quantity cfeDNA release, and thus more accurate results from aneuploidy testing. Some have suggested that cfeDNA present in spent embryo culture medium or blastocoel fluid also has the potential to be used for monogenic disorder testing (Capalbo et al., 2018; Wu et al., 2015; Madjunkova et al., 2018).

However, several very important issues still need to be addressed before routine clinical application of NIPGT. These include: minimization of maternal DNA contamination risk, determining the factors affecting accuracy, and optimization of the WGA protocol for cfeDNA.

In this example, the accuracy and reliability is assessed for utilizing cfeDNA in SEM+BF samples for blastocyst chromosomal status detection in comparison to corresponding TE biopsy samples in a larger cohort of fresh cultured embryos as compared with previous approaches, such as described by Kuznyetsov et al., 2018. Factors that could influence this method include: 1) quantity of amplified cfeDNA obtained; 2) the effect of blastocyst morphological grades on cfeDNA; 3) average size of WGA-DNA fragments from good and moderate/low quality blastocysts, and 4) whole genome amplification of cfeDNA from SEM+BF samples with or without a cell lysis/extraction enzymatic step.

### Materials & Methods

Ethics approval. This research received approval from the University of Toronto Research Ethics Board (IRB #30251). Informed consent was obtained for all patients included in this study. All experiments were performed in accordance with the relevant guidelines and regulations.

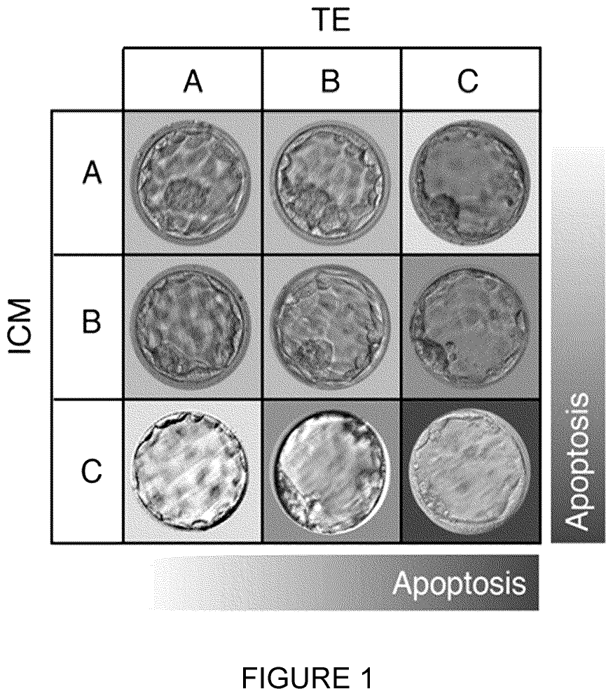

Patients and Samples. Combined spent embryo culture medium and blastocoel fluid samples (NIPGT) from a total of 145 fresh blastocysts and their corresponding trophectoderm (TE) biopsy samples were analyzed for this report. These samples were from 28 patients, aged 33 to 42 years (mean 36.8+/−3.0 years) undergoing PGT-A cycles from October 2018 to January 2019 at the CReATe Fertility Centre, Toronto, Canada. Of these, 102 NIPGT samples and their corresponding TE biopsy samples were used to assess the impact of static embryo morphology on efficacy and accuracy of NIPGT-A. Morphology of these blastocysts was evaluated based on the SART scoring system (Heitmann et al., 2013) with small modifications. In this system, grade 1 are fully expanded blastocysts and grade 2 are expanding blastocysts. Good quality embryos are considered 1313 (i.e. AA, AB, BA, or BB) (n=55), and moderate/low quality are <BB (i.e. AC, CA, BC, CB, or CC) (n=47) (FIG. 1). For the 102 fully expanded blasts from 28 patients, the ages ranged from 29-40 years (35.0±3.3).

To evaluate the reliability, efficacy and accuracy of the SurePlex™ whole genome amplification (WGA) system and WGA with vs. without a cell-lysis step, two aliquots of combined spent embryo culture medium and blastocoel fluid and corresponding TE biopsies from the remaining 43 blastocysts were included.

FIG. 1 shows amplified morphological scoring of blastocyst categorized them into two groups: good quality (≥BB) and average/low quality (<BB). The rate of apoptotic events is higher in lower morphologic grade embryos.

Embryo culture. After collection of all cumulus-oocyte complexes, the cumulus and corona radiata cells were removed by a combination of enzymatic and mechanical (pipetting) procedures. Mature metaphase II oocytes were fertilized by intracytoplasmic sperm injection (ICSI). Following ICSI, each oocyte was placed in a culture dish containing 25 μl Sage1-Step™ medium with serum protein supplement (Origio, Denmark) under oil and then placed into the incubator (K Systems G210, Cooper Surgical, USA). Laser zona opening (zona breach) was performed on day 4 to facilitate passage of embryonic cfeDNA into the culture media. Each laser zona-opened embryo was transferred on day 4 to fresh 20 μl Sage1-Step™ medium with serum protein supplement (Origio, Denmark) and cultured until blastocyst formation. The day 1 wash and the day 4 zona breach was performed for all test results reported.

### Sample Collection

Collection of spent embryo culture media and blastocoel fluid. The non-invasive and invasive preimplantation genetic testing (NIPGT-A and PGT-A) workflow has been described previously (Kuznyetsov et al., 2018; Fuchs Weizman et al., 2019). In brief, when blastocyst full expansion was observed on day 5, 6 or 7, the blastocysts were collapsed by a single laser pulse at the junction of TE cells (infrared Zilos-tk™ or Lykoslaser™, Hamilton Thorne Biosciences, Beverley, MA) allowing release of blastocoel fluid (BF) into the media. After transferring the embryo to a biopsy dish, collection of the mixture of leaked BF together with embryo culture media (˜5 μL) as one NIPGT sample was done using sterile single use pipettes in sterile RNase-DNase-free PCR tubes and stored at −80° C. until analyzed. Control blank media samples were cultured under the same conditions and served as negative controls.

Trophectoderm cells (TE). A corresponding TE biopsy sample from each embryo was obtained using previously described protocols (Kuznyetsov et al., 2018; Fuchs Weizman et al., 2019). All blastocysts were transferred to a biopsy dish containing 20 μL media under oil. Four to six trophectoderm cells were biopsied from each blastocyst. The biopsied cells were placed immediately in RNase-DNase-free PCR tubes and stored at −80° C. until analyzed. Control blank media samples were collected as negative controls.

Whole genome amplification, sequencing, and analysis. Whole genome amplification (WGA) was performed, according to manufacturer's instructions, using the SurePlex™ WGA (VeriSeq™ PGS Kit, Illumina). The WGA starts with enzymatic lysis of biopsied cells to release gDNA followed by a pre-amplification and amplification steps using degenerative primers for uniform random whole genome amplification. WGA samples were analyzed using Qubit3.0™ Fluorimeter to assess the double stranded DNA concentration. All samples were diluted to 0.2 ng/μl and a total of 1 ng from each sample was tagmented and amplified using random primers. The kit contains 24 unique indexes added by amplification. Indexed DNA libraries were cleaned-up (AMPure™ XP beads 1:1 ratio) and normalized using magnetic beads. The normalized libraries were pooled, denatured, and sequenced using a MiSeq™ (single-end, 1×36 bp). Alignment and demultiplexing are done as part of the VeriSeq PGS protocol on MiSeq and CNV analysis and visualization were done using BlueFuse™ Multi (Illumina) software.

Assessment of embryonic and cell free embryonic DNA. WGA products (SurePlex™ kit, Illumina) were quantified with the Qubit3.0-Fluorometer and their size distribution was assessed using 2100 BioAnalyzer™ (DNA high sensitivity chip, Agilent).

Statistical analysis. The concordance rate for whole chromosome copy number abnormalities between NIPGT samples and corresponding TE biopsy PGT-A samples were analysed. Results were statistically evaluated using Chi-squared and Fisher's exact testing, with significance at p<0.05.

### Results

The blastocyst morphology had no effect on cfeDNA quantity and the mean size of WGA-DNA fragments in NIPGT samples.

Table 1 shows analysis of the amount of amplified DNA and fragment sizes from each of the samples. The amount was highest in TE biopsy samples (˜4×). The amount of amplified cfeDNA derived from good quality blastocysts was not significantly different than that from moderate/low quality blastocysts. Blank medium negative control samples associated with each sample that underwent WGA showed no amplification in all cases. The average size of WGA-DNA fragments from NIPGT samples from good quality blastocysts (n=55) vs. from moderate/low quality blastocysts (n=47) was not statistically different (Table 1).

TABLE 1
Blastocyst Quality, And Amount And Size Of Amplified Nuclear DNA In NIPGT Samples

| | | | | | Average size of | | |
|---|---|---|---|---|---|---|---|
| Types of | Amplification | | WGA-DNA | WGA-DNA | WGA-DNA | Informative NGS | |
| samples | rate (%) | | range (ng/μl) | amount (ng/μl) | fragments | results (%) | |
| NIPGT, ≥BB | 55/55 | (100) | 5.1 to 30.0 | 14.6 ± 4.9* | 735.6 ± 27.1 bp* | 48/55 | (87.3)* |
| NIPGT, <BB | 47/47 | (100) | 6.3 to 36.0 | 15.9 ± 6.0* | 746.9 ± 42.6 bp* | 42/47 | (89.4)* |
| NIPGT, total | 102/102 | (100) | 5.1 to 36.0 | — | — | 90/102 | (88.2)* |
| TE biopsy | 102/102 | (100) | 25.0 to 46.0 | 31.3 ± 1.2 | 820.0 ± 32.5 bp | 100/102 | (98.0)** |

Blastocyst morphology had no effect on the rate of NIPGT-A informative results or concordance compared to standard PGT-A.

Informative next-generation sequencing (NGS) results (Table 2) were obtained for 98.0% of TE biopsies and for 88.2% of NIPGT samples (87.3% for good quality and 89.4% for moderate/low quality blastocysts; p>0.05). The overall concordance rate per sample for whole chromosome copy number abnormalities for euploidy/aneuploidy status between NIPGT and TE biopsy samples was 88/90 (97.8%), and was not different between good 47/48 (97.9%) and moderate/low quality blastocysts 41/42 (97.9%) (p>0.05) (Table 2 and Table 3). NIPGT-A analysis correlated with PGT-A results for gender (100%) and aneuploidy in 92.6% of NIPGT samples. Aneuploidy/euploidy concordance rate did not depend on blastocyst quality (Table 2).

TABLE 2
Blastocyst Quality And Concordance Rate For Whole Chromosome Copy Number
Abnormalities Between NIPGT Samples And Corresponding TE Biopsy Samples

| | Ploidy status (%) | | | | | Euploid | | Aneuploid | |
|---|---|---|---|---|---|---|---|---|---|
| Type of samples | Per sample | | Per chromosome | | Gender (%) | samples (%) | | samples (%) | |
| NIPGT, ≥BB vs. TE | 47/48 | (97.9)* | 1128/1152 | (97.9)* | 48/48 (100) | 37/38 | (97.4)* | 10/11 | (90.9)* |
| NIPGT, <BB vs. TE | 41/42 | (97.6)* | 984/1008 | (97.6)* | 42/42 (100) | 26/26 | (100)* | 15/16 | (93.8)* |
| NIPGT, total vs. TE | 88/90 | (97.8) | 2112/2160 | (97.8)* | 90/90 (100) | 63/64 | (98.4) | 25/27 | (92.6) |

Ploidy status - euploid or aneuploidy;
Aneuploid - whole/segmental chromosome aneuploidy
*Not statistically significant

A detailed summary of NIPGT-A and PGT-A results for paired samples of embryos with good and moderate/poor morphology are presented in Table 3. NGS results are presented from all trophectoderm biopsy and NIPGT samples obtained from the corresponding blastocyst. Blastocysts were grouped based on their static morphology in good if graded as ≥1/2BB, or average/poor if graded <1/2BB.

TABLE 3
NGS Results From All Trophectoderm Biopsy And NIPGT
Samples Obtained From The Corresponding Blastocyst

| | | |
|---|---|---|
| Embryo No. | TE biopsy | NIPGT, ≥BB |

Euploid-euploid

| | | |
|---|---|---|
| 1 | XX; normal | XX; normal |
| 2 | XX; normal | XX; normal |
| 3 | XX; normal | XX; normal |
| 4 | XX; normal | XX; normal |
| 5 | XX; normal | XX; normal |
| 6 | XX; normal | XX; normal |
| 7 | XX; normal | XX; normal |
| 8 | XY; normal | XY; normal |
| 9 | XX; normal | XX; normal |
| 10 | XX; normal | XX; normal |
| 11 | XY; normal | XY; normal |
| 12 | XY; normal | XY; normal |
| 13 | XX; normal | XX; normal |
| 14 | XY; normal | XY; normal |
| 15 | XY; normal | XY; normal |
| 16 | XY; normal | XY; normal |
| 17 | XY; normal | XY; normal |
| 18 | XX; normal | XX; normal |
| 19 | XY; normal | XY; normal |
| 20 | XY; normal | XY; normal |
| 21 | XX; normal | XX; normal |
| 22 | XY; normal | XY; normal |
| 23 | XY; normal | XY; normal |
| 24 | XX; normal | XX. normal |
| 25 | XY; normal | XY; normal |
| 26 | XX; normal | XX; normal |

Euploid-mosaic

| | | |
|---|---|---|
| 27 | XY; normal | XY; mosaic −8 (70%) |
| 28 | XY; mosaic −9 (30%) | XY; normal |
| 29 | XX; mosaic −15 (20%) | XX; normal |
| 30 | XX; mosaic: −8p (35%), −9p (35%) | XX; normal |
| 31 | XX; normal | XX; mosaic −9 (70%) |

Mosaic-mosaic

| | | |
|---|---|---|
| 32 | XY; mosaic −4q (118.6 Mb, 20%) | XY; mosaic +13q21.1 ± 31.3 |
| | | (34.84 Mb, 60%) |
| 33 | XY; mosaic loss: (−4q22.1-q35.2, | XY; mosaic gain: |
| | 100 Mb, 20%)* | (+4q 22.1-q35.2, 100 Mb, 30%)* |
| 34 | XY; mosaic: +8q (50%), −5q15-q35.3 | XY; mosaic −3 (70%) |
| | (88 MB, 35%) | |
| 35 | XY; mosaic −16 (60%) | XY; mosaic −17 (−70%) |
| 36 | XX; mosaic: +8 (50%), +19 (50%) | XX; mosaic: −17 (30%) |
| 37 | XX; mosaic: −22 (60%) | XX; mosaic: −22 (70%) |

Segmental aneuploid-euploid

| | | |
|---|---|---|
| 38 | XX; normal | XX; −5q23.3-q35.3 (51.71 Mb) |

Aneuploid-aneuploid

| 39 | XX; −14 | XX; −14 |
|---|---|---|
| 40 | XX; −15 | XX; −15 |
| 41 | XY; +16, +21 | XY; +16, +21 |
| 42 | XX; +16 | XX; +16 |
| 43 | XY; −7, −15, −18 | XY; −7, −15, −18 |
| 44 | XX; −22 | XX; −22 |
| 45 | XX; −13 | XX; −13 |
| 46 | XY; +16 | XY; +16 |
| 47 | XY; −7 | XY; −7 |
| 48 | XY; +21 | XY; +21 |
| Embryo No. | TE biopsy | NIPGT, <BB |

Euploid-euploid

| | | |
|---|---|---|
| 1 | XX; normal | XX; normal |
| 2 | XY; normal | XY; normal |
| 3 | XY; normal | XY; normal |
| 4 | XX; normal | XX; normal |
| 5 | XY; normal | XY; normal |
| 6 | XX; normal | XX; normal |
| 7 | XX; normal | XX; normal |
| 8 | XX; normal | XX; normal |
| 9 | XY; normal | XY; normal |
| 10 | XX; normal | XX; normal |
| 11 | XX; normal | XX; normal |
| 12 | XY; normal | XY; normal |
| 13 | XY; normal | XY; normal |
| 14 | XX; normal | XX; normal |
| 15 | XY; normal | XY; normal |
| 16 | XX; normal | XX; normal |
| 17 | XX; normal | XX; normal |

Euploid-mosaic

| | | |
|---|---|---|
| 18 | XX; mosaic loss: (−3p.26.3 ± p25.2, | XX; normal |
| | 12 Mb, 35%) | |
| 19 | XX; mosaic gain: +12p13.33-q23.3 | XX; normal |
| | (105.5 Mb, 45%) | |
| 20 | XY; mosaic +16p (40%) | XY; normal |
| 21 | XX; mosaic −8 (40%) | XX; normal |

Mosaic-mosaic

| | | |
|---|---|---|
| 22 | XY; mosaic +6q22.1-q.25.2 (38.5 Mb, | XY; mosaic −8 (50%) |
| | 45%), mosaic −15 (50%) | |
| 23 | XY; mosaic loss: −3q26.31-q29 | XY; mosaic loss: −3q26.31-q29 |
| | (24.4 Mb, 65%) | (24.4 Mb, 65%) |
| 24 | XX; mosaic: +4 (30%), +5 (30%), +8 | XX; mosaic: +5 (40%), +10 (40%) |
| | (30%), +10 (50%) | |
| 25 | XY; mosaic +14q11.2-q22.1, | XY; mosaic −14q11.2-q32.33, |
| | 33.1 Mb, 65%* | 84.69 Mb, 60%* |
| 26 | XY; mosaic +4 (40%) | XY; mosaic +4 (50%) |

Complex Aneuploid-mosaic

| | | |
|---|---|---|
| 27 | XX; −18 | XX; mosaic −18q12.2-q23 |
| | | (43.75 Mb, 40%) |

Aneuploid-aneuploid

| | | |
|---|---|---|
| 28 | XY; −5, −13 | XY; −5, −13 |
| 29 | XX; +19 | XX; +19 |
| 30 | XY; +10, −11, −20 | XY; +10, −11, −20 |
| 31 | XY; +11, −16, +22 | XY; +9, +11, −16, +22 |
| 32 | XX; +22 | XX; +22 |
| 33 | XX; +13, +19, −21 | XX; +13, +19, −21 |
| 34 | XY; +11, mosaic gain: +10q23.31-26.3 | XY; +10, +11, +16 |
| | (44.26 MB, 50%) | |
| 35 | XX; −3p26.3-p22.1 (39, 8 Mb) | XX; −3p26.3-p22.1 (39, 8 Mb) |
| 36 | XY; −17, +21, mosaic: +1p31.1-p21.1 | XY; −17, +21 |
| | (39 Mb, 30%) | |
| 37 | XY; −19 | XY; −19 |
| 38 | XY; −4, −8, +9, +18 | XY; −4, −8, +9, +18 |
| 39 | XY; +22 | XY; +22 |
| 40 | XX; −11 | XX; −11 |
| 41 | XY; −22 | XY; −22 |
| 42 | XY; +16 | XY; +16 |

*Mosaic-complementary in terms of chromosomal gain versus loss between TE biopsy and NIPGT samples

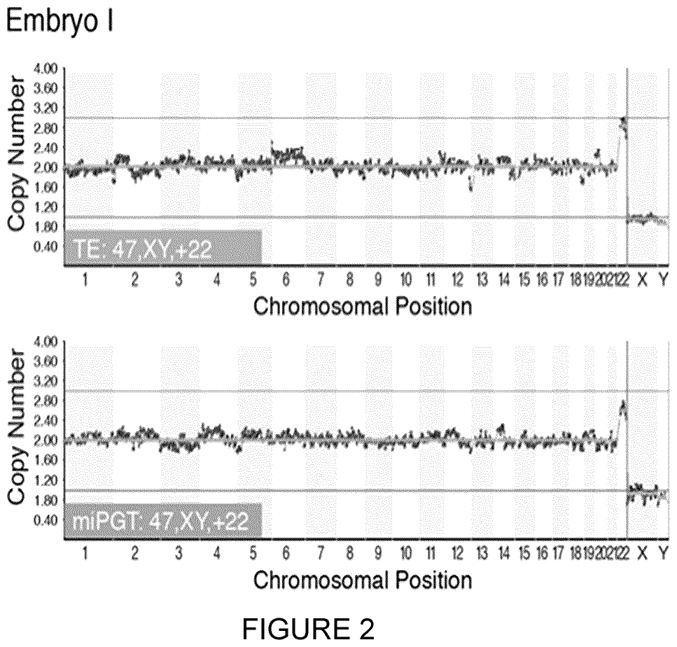

FIG. 2-FIG. 5 present four examples of NGS results representing 24 chromosome copy number (CNV) plots from TE biopsy and corresponding NIPGT samples with concordant results for aneuploidy in three examples and mosaic-complementary in terms of loss versus gain on chromosome 4q (segmental chromosomal mosaicism) in the fourth embryo.

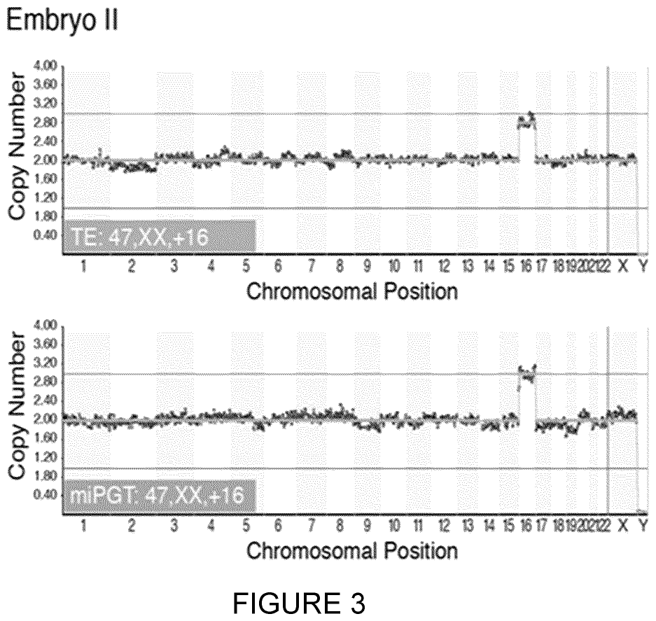

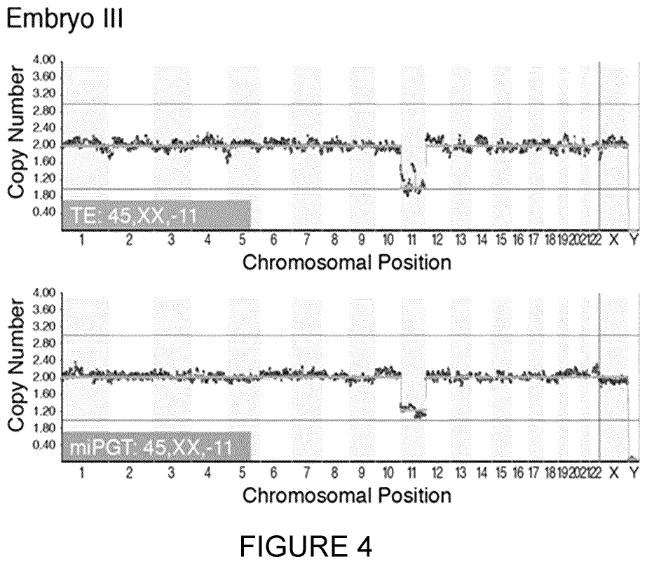

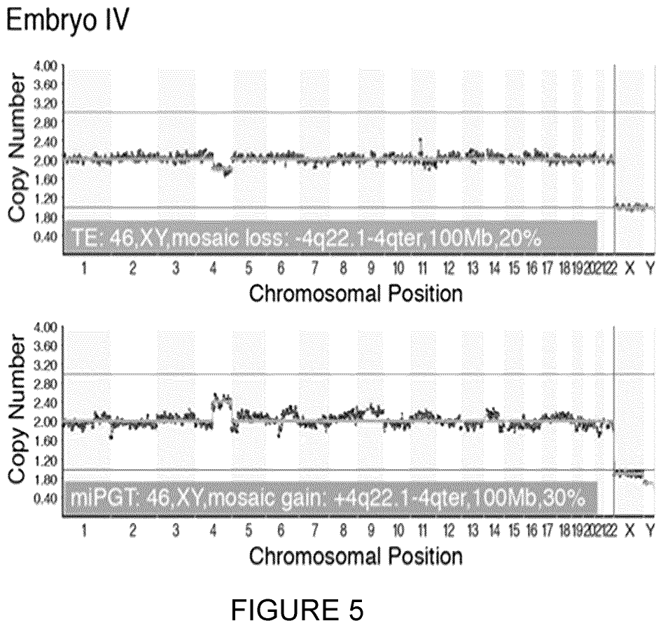

FIG. 2 depicts copy number based per chromosomal position for Embryo I (TE: 47, XY, +22, upper panel; miPGT: 47, XY, +22, lower panel). FIG. 3 depicts copy number per chromosomal position for Embryo II (TE: 47, XX, +16 upper panel; miPGT: 47, XX, +16, lower panel). FIG. 4 depicts copy number per chromosomal position for Embryo III (TE: 45, XX, −11, upper panel; miPGT: 45, XX, −11, lower panel). FIG. 5 depicts copy number per chromosomal position for Embryo IV (TE: 46, XY, mosaic loss: −4q22.1 −4qter, 100 Mb, 20%, upper panel; miPGT: 46, XY, mosaic gain: +4q22.1 −4qter, 100 Mb, 30%, lower panel).

Mosaicism results were complex when comparing PGT-A with NIPGT-A (Table 3). There were 4 cases in which there was relatively full concordance between PGT-A with NIPGT-A in terms of mosaicism and the chromosome involved. In one case (embryo #33, Table 3), there was a mosaic segmental loss on chromosome 4q in the TE sample versus a complementary gain on chromosome 4q in the NIPGT sample. There were 4 other cases that showed mosaicism in both PGT-A and NIPGT-A samples but there was discordance as to which chromosome was involved. Interestingly, there were 3 cases in which NIPGT-A showed euploidy and the PGT-A showed mosaicism, and 2 cases in which the PGT-A showed euploidy and the NIPGT-A showed mosaicism (Table 3). No obvious difference was seen in the rate of mosaicism detected or discordance rate between PGT-A and NIPGT-A for good quality vs medium/low quality embryos, but the numbers of mosaic cases in these two cohorts was too small to finalize conclusions regarding this comparison.

DNA amplification rate, amount of amplified cfeDNA and NGS results from NIPGT samples with or without cell lysis/extraction enzyme step.

The second objective of this Example was to determine the accuracy, efficacy and reliability of whole genome amplification to determine ploidy status of the blastocyst using combined SEM+BF samples with or without using the cell lysis/extraction enzyme step before WGA on separate aliquots from the same pool of SEM+BF collected for NIPGT-A analysis.

Two aliquots were collected from SEM+BF (NIPGT samples) (n=86) from 43 additional blastocysts. The amount of amplified DNA and NGS data was analysed from the 86 NIPGT samples and corresponding 43 trophectoderm biopsy samples obtained from fresh blastocysts that underwent PGT-A cycles from. The first aliquot (NIPGT-1) SEM+BF sample followed the standard SurePlex WGA protocol which starts with 5 μl of sample and a cell lysis step. The second aliquot from the same SEM+BF pool (NIPGT-2) was amplified following a modified WGA SurePlex protocol that starts with 10 μl of sample and the direct pre-amplification. WGA products of NIPGT-1 and NIPGT-2 samples were compared with each other, and results were compared with the corresponding trophectoderm biopsy sample used as a control.

The amount of concentrated amplified cfeDNA from NIPGT-1 samples was higher than in NIPGT-2 samples, however this was not statistically significant. Respective blank medium (negative control) associated with each sample showed no amplification in all cases, as seen in Table 4, which shows the amount of concentrated amplified nuclear DNA in NIPGT-1 (WGA with cell lysis) and NIPGT-2 (WGA without cell lysis) samples.

TABLE 4
Amount of Concentrated Amplified Nuclear DNA
in NIPGT-1 Samples (WGA with Cell Lysis) and
NIPGT-2 Samples (WGA Without Cell Lysis)

| | Amplifi- | WGA-DNA | WGA-DNA | Informative | |
|---|---|---|---|---|---|
| Types of | cation rate | range | concentration | NGS results | |
| samples | (%) | (ng/μl) | (ng/μl) | (%) | |
| NIPGT-1 | 43/43 (100) | 6.3 to 85.9 | 37.3 ± 19.4* | 40/43 | (93.0)* |
| NIPGT-2 | 43/43 (100) | 10.2 to 72.7 | 32.8 ± 16.3* | 38/43 | (88.4)* |
| NIPGT, | 86/86 (100) | 6.3 to 85.9 | — | 78/86 | (90.7) |
| total | | | | | |
| TE biopsy | 43/43 (100) | — | — | 41/43 | (95.3)* |

*Not statistically significant

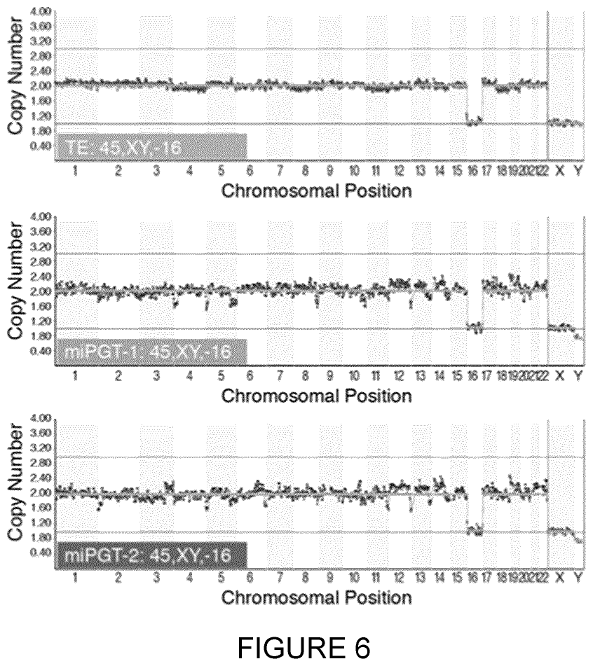

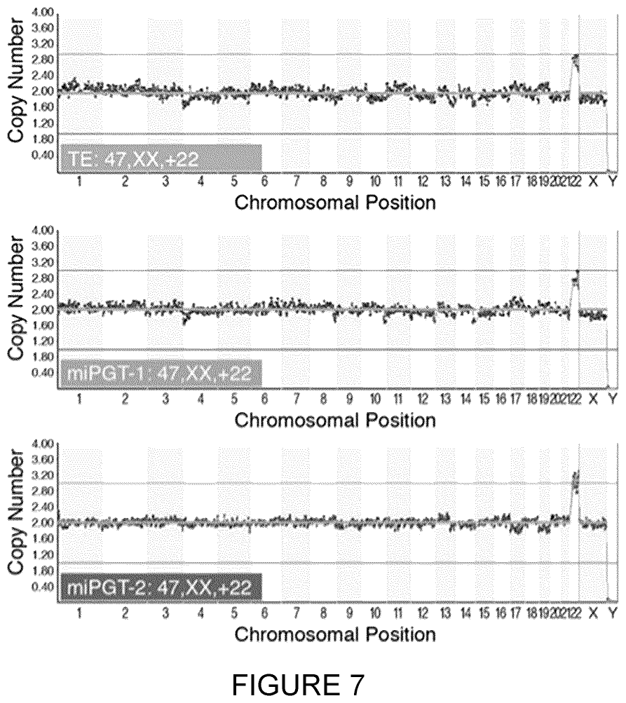

Table 5 shows informative NGS results, as obtained for 95.3% trophectoderm biopsies, for 93.0% of NIPGT-1, and for 88.4% of NIPGT-2 samples; the difference was not statistically significant. There was a high concordance rate per sample for whole chromosome copy number abnormalities between: 1) NIPGT-1 and TE biopsy samples (97.4%), 2) NIPGT-2 and TE biopsy samples (97.2%) and 3) NIPGT-1 and NIPGT-2 samples (100%). NIPGT correctly determined the gender of the embryos and aneuploidy for all chromosomes in non-invasive samples (see FIG. 6 and FIG. 7). Aneuploid embryo #43 had complimentary aneuploidy, in term of gain versus loss of chromosome 9, between TE biopsy and both NIPGT samples. Interestingly, analysis of the aneuploid embryo #40 showed that the TE sample was trisomy 13, while both NIPGT-1 and NIPGT-2 had monosomy 20. A test of the inner cell mass and rest of the TE was not able to be conducted for these embryos, absent patient consent for donation of the embryo to research in order to perform a second biopsy. The concordance rate for whole chromosome copy number abnormalities between corresponding NIPGT-1 samples (WGA with cell lysis), NIPGT-2 samples (WGA without cell lysis) and TE samples are provided.

FIG. 6 and FIG. 7 provide example of NGS results from TE biopsy and corresponding NIPGT-1 (WGA with cell lysis) and NIPGT-2 (WGA without cell lysis) samples. FIG. 6 depicts a copy number per chromosomal position for Embryo a (TE: 45, XY, −16, upper panel; miPGT-1: 45, XY, −16, middle panel; miPGT-2: 45, XY, −16, lower panel), while FIG. 6 depicts a copy number per chromosomal position for Embryo b (TE: 47, XX, +22, upper panel; miPGT-1: 47, XX, +22, middle panel; miPGT-2: 47, XX, +22, lower panel).

TABLE 5
Concordance Rate for Whole Chromosome Copy Number Abnormalities Between Corresponding
NIPGT-1 (WGA with cell lysis), NIPGT-2 (WGA without cell lysis) and TE Samples.

| | Ploidy status (%) | | | | | | | | |
|---|---|---|---|---|---|---|---|---|---|
| | | | Per | | | Euploid | | Aneuploid | |
| Type of samples | Per sample | | chromosome | | Gender (%) | embryos (%) | | embryos (%) | |
| NIPGT-1 vs. TE | 37/38 | (97.4)* | 888/912 | (97.4)* | 38/38 (100) | 23/24 | (95.8)* | 14/15 | (93.3)* |
| NIPGT-2 vs. TE | 35/36 | (97.2)* | 840/864 | (97.2)* | 36/36 (100) | 22/23 | (95.7)* | 13/14 | (92.9)* |
| NIPGT-1 vs. | 38/38 | (100)* | 912/912 | (100)* | 38/38 (100) | 24/24 | (100)* | 14/14 | (100)* |
| NIPGT-2 | | | | | | | | | |

Ploidy status - euploid or aneuploidy;
Aneuploid - whole/segmental chromosome aneuploidy
*Not statistically significant

Table 6 shows a summary of NGS results from chromosomal copy number analysis from NIPGT-1 (WGA with cell lysis), NIPGT-2 (WGA without cell lysis) and TE biopsy samples obtained from the same blastocyst.

TABLE 6
NGS Results from Chromosomal Copy Number Analysis from NIPGT-1,
NIPGT-2, and TE Biopsy Samples from the Same Blastocyst

| | | | WGA-DNA | | WGA-DNA |
|---|---|---|---|---|---|
| Embryo | TE biopsy | NIPGT-1 | (ng/μl)* | NIPGT-2 | (ng/μl)* |
| 1 | XX; −13 | XX; −13 | 32.3 | XX; −13 | 36.2 |
| 2 | XY; +11, | XY; +10, +11, +16 | 52.4 | XY; +10, +11, +16 | 19.5 |
| | mosaic: +10q23.31-q26.3 | | | | |
| | (44.3 Mb, 50%) | | | | |
| 3 | XX; normal | XX; normal | 85.9 | XX; normal | 28.7 |
| 4 | XX; normal | XX; normal | 32.9 | Inconclusive | 47.8 |
| 5 | XY; −19 | XY; −19 | 36.2 | XY; −19 | 43.9 |
| 6 | XX; −3p26.3-p22.1 | Inconclusive | 45.0 | Inconclusive | 13.2 |
| | (39.8 Mb) | | | | |
| 7 | XX; mosaic −8 (40%) | XX; normal | 47.3 | XX; normal | 34.4 |
| 8 | XY; −17, +21, | XY; −17, +21 | 29.2 | XY; −17, +21 | 40.0 |
| | mosaic: +1p31.1-p.21.1 | | | | |
| | (39 Mb, 30%) | | | | |
| 9 | XY; normal | XY; normal | 41.4 | XY; normal | 28.2 |
| 10 | XY; normal | XY; normal | 31.4 | XY; normal | 14.1 |
| 11 | XX; normal | Chaotic DNA | 19.7 | Chaotic DNA | 10.2 |
| | | signal | | signal | |
| 12 | XY; mosaic: −16 (60%) | XY; mosaic: −16 | 32.9 | XY; mosaic: −16 | 14.7 |
| | | (70%) | | (50%) | |
| 13 | XY; mosaic: −1 (40%), −6 | XY; normal | 31.5 | XY; normal | 29.2 |
| | (40%) | | | | |
| 14 | XY; +16 | XY; +16 | 28.8 | XY; +16 | 46.1 |
| 15 | XX; normal | XX; normal | 18.1 | XX; normal | 12.8 |
| 16 | XX; normal | XX; normal | 34.1 | XX; normal | 36.9 |
| 17 | XX; normal | XX; normal | 42.9 | XX; normal | 41.2 |
| 18 | XY; −16 | XY; −16 | 73.1 | XY; −16 | 67.7 |
| 19 | XY; normal | XY; normal | 48.3 | XY; normal | 38.0 |
| 20 | XX; normal | Inconclusive | 40.7 | Inconclusive | 22.2 |
| 21 | XX; normal | XX; mosaic | 69.7 | XX; mosaic | 60.4 |
| | | segmental: +1q21.2-q32.1 | | segmental: +1q21.2-q32.1 | |
| 22 | XY; normal | XY; normal | 34.1 | XY; normal | 17.2 |
| 23 | Chaotic DNA signal | XY; +22 | 33.9 | XY; +22 | 32.9 |
| 24 | XY; normal | XY; normal | 79.1 | XY; normal | 60.5 |
| 25 | XY; normal | XY; normal | 67.2 | XY; normal | 45.8 |
| 26 | XY; +19 | XY; +19 | 80.1 | XY; +19 | 72.7 |
| 27 | XX; +22 | XX; +22 | 51.9 | XX; +22 | 34.2 |
| 28 | XX; normal | XX; normal | 18.5 | XX; normal | 14.7 |
| 29 | XX; −19; mosaic −16 | XX; +18; mosaic −16 | 39.5 | XX; +18; mosaic −16 | 31.3 |
| | (40%), −18 (20%) | (70%), −19 (60%) | | (70%), −19 (60%) | |
| 30 | XY; normal | XY; normal | 15.7 | XY; normal | 15.7 |
| 31 | XY; +21 | XY; +21 | 16.6 | XY; +21 | 29.9 |
| 32 | XY; normal | XY; normal | 37.7 | XY; normal | 39.5 |
| 33 | Chaotic DNA signal | XY; normal | 17.7 | XY; normal | 18.4 |
| 34 | XX; normal | XX; normal | 31.3 | XX; normal | 30.9 |
| 35 | XY; normal | XY; normal | 16.1 | XY; normal | 17.2 |
| 36 | XY; −22 | XY; −22 | 17.7 | XY; −22 | 18.5 |
| 37 | XY; −8q; Mosaic | XY; −8q; +8p | 45.8 | XY; −8q; +8p | 38.6 |
| | (−8p, 35%) | | | | |
| 38 | XY; mosaic −22 (30%) | XY; mosaic −22 (30%) | 23.9 | XY; normal | 19.4 |
| 39 | XX; +22 | XX; normal | 31.2 | XX; normal | 22.8 |
| 40 | XX; +13 | XX; −20 | 6.8 | XX; −20 | 12.1 |
| 41 | XX; +13 | XX; +13 | 6.3 | Inconclusive | 33.1 |
| 42 | XY; normal | XY; normal | 30.5 | XY; normal | 61.4 |
| 43 | XX; +9** | XX; −9** | 28.4 | XX; −9** | 56.4 |

*Concentrated WGA-DNA
**Aneuploid-complementary in term of gain versus loss of chromosome 9 between TE biopsy and both NIPGT samples

### Discussion

It has been recently reported that only ˜8% of DNA in spent embryo culture medium is embryonic in origin (Vera-Rodriquez et al., 2018; Hammond et al., 2016). This could potentially impact the analysis of embryonic DNA from spent culture media. To minimize maternal contamination in our studies, we modified the procedure steps during Day 0 to Day 4 of embryo culture to include careful removal of residual corona cells by pipetting and washing (Kuznyetsov et al., 2018). Using a fluorescently labelled short tandem repeat (STR) marker for the analysis of both embryonic DNA (TE cells) and NIPGT samples, this example confirms that this step minimizes maternal contamination, and is important for avoiding misdiagnosis of NIPGT-A.

In addition, the approach described herein to transfer embryos into individual fresh droplets of medium on Day 4 and using SEM+BF samples obtained after Day 5/6 culture, promotes a yield of less degraded cfeDNA. This method considers the embryonic genome activation stage in human embryos (Dillon et al., 2015; Galan et al, 2010) and the number of blastomeres on Day 4 versus Day 3. Huang et al. (2019) and Rubio et al. (2019) showed the superiority of using spent embryo fresh culture media from embryos cultured from day 4 to day 5/6 for non-invasive PGT-A, compared to samples collected from a more extended culture period, which seems to result in a more degraded cfeDNA sample.

The issue of whether nucleic acids can penetrate though the zona pellucida is an important consideration. In this example, it was hypothesized that assisted hatching (AH) may facilitate the release of a high molecular weight cfeDNA from BF into the culture medium. In this example, AH was performed on Day 4. For better passage of embryonic DNA, some researchers have tried to use zona opening on Day 3 (Shamonki et al., 2016; Vera-Rodriguez, M. et al., 2018). In other studies assisted hatching was not performed prior to conducting the TE biopsy (Rubio et al., 2019). Ho et al. (2018) found that assisted hatching on Day 3 did not influence cfeDNA concentration or accuracy of cfeDNA sequencing for aneuploidy screening. CfeDNA isolated from spent embryo culture medium on Day 2/3 has a low molecular weight, where cellular fragmentation may also play a role (Stigliani et al., 2013). Vera-Rodriguez et al. (2018) pointed out that samples isolated from spent embryo culture medium could contain high molecular weight DNA or sheared DNA. In this example, the amount of cfeDNA in D4-D5/6 embryo culture media was not measured, with or without using laser zona opening on Day 4, to test this hypothesis.

The mechanism(s) underlying the release of embryonic DNA into the culture medium remain unclear and the origin of these DNA fragments is unknown (Munne et al., 2018; Battablia et al., 2019). One possibility is that nucleosome sized DNA fragments (˜180-200 bp) are being released from cells as a result of apoptosis (Handyside, 2016; Xu et al, 2016; Hammond et al, 2016; Rule et al., 2018). Another mechanism that could contribute cell free DNA with longer sized DNA fragments is necrosis (Hammond et al., 2016).

Previous studies have reported that the concentration of cfeDNA correlates with apoptotic events (Rule et al., 2018). In this example, it was hypothesized that lower grade blastocysts, which tend to have a higher rate of apoptosis, will release a higher quantity of cfeDNA into the medium. Ho et al. (2018) found that blastocyst morphology did not influence cfeDNA concentration in spent embryo culture medium or accuracy for aneuploidy screening. Rule et al., (2018) indicated that cfeDNA in blastocoel fluid positively correlates with a high embryonic morphology score, which suggests that the better the embryo morphology, the higher the cfDNA concentration.

The results of our study, which represents the largest number of blastocysts tested using spent embryo culture medium combined with blastocoel fluid, demonstrated that the morphological grade of blastocysts does not affect the rate of informative NGS results from cfeDNA. The amount of amplified DNA from good quality blastocysts was slightly lower than that from moderate/low quality blastocysts, however the difference was not statistically significant. The concordance rate per sample for whole chromosome copy number between NIPGT and TE biopsy samples (see Table 2 and Table 3), for both good and moderate/low quality blastocysts, was not statistically different. The mean size of WGA-DNA fragments derived from NIPGT samples from good quality blastocysts and from moderate/low quality blastocysts was not statistically different (see Table 1).

Considering the amount of NIPGT-A WGA-DNA from different blastocysts and the size of NIPGT-A WGA-DNA fragments (not close to nucleosomal size), cell apoptosis may not be the only mechanism for DNA release from the ICM and TE into BF and SEM. Therefore, other mechanisms for release of embryonic DNA are probably involved. One possibility is that embryonic DNA in culture media may derive from cells damaged due to the laser pulses used during artificial shrinkage, although the single laser pulse was used at the junction of TE cells located far away from the inner cell mass. Extrachromosomal microDNAs could also be a source of cfeDNA in spent culture medium. Production of these microDNAs is a part of normal cellular physiology, linked to transcriptional activity and mismatch repair. Extrachromosomal microDNAs vary in size from 60 to 2000 base pairs. They are abundant in all tissue types of mammalian cells, including sperm (Dillon et al., 2015). In contrast to accumulation of embryonic DNA in culture medium by apoptosis or necrosis, this mechanism would not necessarily depend on cell death.

Functional aspect of the DNA or RNA released by the developing preimplantation embryo is unknown and whether it is involved in cellular communication is a subject of research. Extracellular vesicles (EVs) in blastocoel fluid and embryo culture medium as a transport vehicle may contain packed DNA to transmit information between the cells of trophectoderm and inner cell mass (Hammond et al., 2016). Similar cross-talk by means of EVs transferring miRNAs and other molecules (mRNAs, DNA, lipids and proteins) has been described among cells. EVs may be able to traverse the zona pellucida when human embryos are cultured in vitro, which supports this hypothesis (Vyas et al., 2019).

Since cell-free embryonic DNA consists of relatively short DNA fragments, the analysis of spent culture medium requires modifications of the standard WGA protocol. In this example, it was hypothesized that for cfeDNA analysis, the SurePlex™ whole genome amplification kit can be used without the need for a cell lysis/extraction DNA step. This example illustrates that NIPGT-1 and NIPGT-2 samples (see Table 5 and Table 6) show a similar high concordance rate with corresponding TE biopsy samples for a chromosome copy number. Thus, amplification of cfeDNA without using the cell lysis/extraction DNA step may reduce the risk of maternal contamination of NIPGT samples by residual cumulus/corona cells.

Assisted hatching is typically performed using a laser pulse prior to blastocyst vitrification, resulting in artificial shrinkage of the blastocoel. This helps to prevent injury from intracellular ice formation and has been shown to improve clinical outcomes Zeng et al., 2018). A single laser pulse creates an opening in the zona pellucida at the cellular junction of trophectoderm cells located far away from the inner cell mass (Magli et al., 2016; Darwish et al., 2016; Mukaida et al., 2016). Therefore, the approach taken in this example to use laser zona opening on Day 4 together with the laser (or microneedle) collapsing of blastocysts prior to TE biopsy for cfeDNA collection should have no negative impact on blastocyst development and does not require an additional laser (or microneedle) collapsing step before blastocyst vitrification as in current clinical practice. Mukaida et al., (2006) describes current clinical practices in this regard.

Collection of both spent embryo culture media and blastocoel fluid as one non-invasive sample increases the quantity and quality of cfeDNA for aneuploidy testing, compared with either spent embryo culture media only, or blastocoel fluid only. In the current example, some WGA-NIPGT DNA samples had chaotic or inconclusive results due to low or poor quality cfeDNA that led to noisy NGS profiles. The same issue was noted by Rubio et al, (2019). Noisy DNA profiles could also be attributed to maternal contamination by residual cumulus or corona cells. Therefore, if used in parallel with TE biopsy, NIPGT-A can improve testing efficacy and accuracy by acting as a backup source of embryonic DNA in cases of inconclusive TE biopsy results, which would also obviate the need for re-biopsy.

Very few studies have compared NIPGT-A samples to corresponding TE biopsy samples and the whole blastocyst, as a gold standard control. Kuznyetsov et al. (2019) as well as Li et al. (2018) and Huang et al. (2019), found concordance rates for both embryo ploidy and chromosome copy number between NIPGT-A samples and whole blastocyst were higher than between TE biopsy and whole blastocyst. Conversely, the study of Ho et al. (2018) showed that the concordance rate for embryo ploidy between NIPGT-A samples and whole blastocyst was lower than between TE biopsy and whole blastocyst. In contrast, Jiao et al. (2019) reported similar concordance between NIPGT-A and TE biopsy samples and between NIGPT-A and the whole blastocyst. Considering results obtained for the non-invasive samples, Huang et al. (2019) also suggested that NIPGT-A is less prone to errors associated with embryo mosaicism and is more reliable than TE biopsy PGT-A.

An embryo transfer of a euploid blastocyst tested by both TE biopsy and cfeDNA from a combined SEM+BF NIPGT-A sample at the CReATe Fertility Centre, Toronto, Canada, resulted in a healthy boy born at full term. This is the first report where two sources of embryonic DNA (SEM+BF and corresponding TE biopsy) were analysed in parallel with clinical PGT-A. In this case, results of cfeDNA analysis from SEM+BF were concordant with the TE biopsy findings. Since that first birth, other pregnancy outcomes have resulted in either all three sources of DNA (removal BF using a microinjection pipette, sampling of SEM and TE biopsy in parallel) were analyzed in a clinical setting on (Ben-Nagi et al., 2019) or two sources of DNA (SEM and TE biopsy in parallel) were analysed (Rubio et al., 2019).

In summary, this example indicates that NIPGT-A, utilizing combined blastocoel fluid and embryo culture medium has advantages and may be superior to TE biopsy for PGT-A for routine clinical use.

### EXAMPLE 2

### NIPGT with Collection And Library Preparation of Spent Culture Media and Blastocoel Fluid to be Used For Non-Invasive Preimplantation Genetic Testing

### Introduction

Preimplantation genetic testing without trophectoderm (TE) biopsy is an attractive approach to avoid any potential risk due to an invasive procedure. Collection of both spent blastocyst culture media and blastocoels fluid as one non-invasive sample can increase the amount and the quality of cell-free embryonic nuclear DNA (cfeDNA) compared with spent embryo culture media only or blastocoel fluid only.

In a clinical setting, limitations may include the very small amount of genetic material available, and the possible contamination from maternal cells. The approach described herein for preparation of oocytes for fertilization involves culturing the embryos from D3 to D4 and collection of the spent media and blastocoel fluid as a one-step procedure.

### Methods

In this Example, a method is described which employs additional steps beyond those described in Example 1. Generally, method steps are conducted as described in Example 1, but include certain modifications to the culturing and library preparation. Differences include, for example, Exo nuclease I treatment is combined with Shrimp Alkaline phosphatase (Exo-SAP-I) to remove single stranded DNA. Further, modified protocol for library prep is employed using NexteraXT™ dual index set A-D (Illumina) that includes increase of amplification cycles from 12 to 16 and sequencing on NextSeq 550 to 0.5-1× genome coverage improves the informative rate of NIPGT samples. Additional details are described below

Embryo culture. After collection of all cumulus-oocyte complexes (COCs), the cumulus and corona radiata are removed by a combination of enzymatic and mechanical (pipetting with stripper) procedures. Mature metaphase II oocytes are fertilized by intracytoplasmic sperm injection (ICSI).

Following ICSI, each oocyte is placed in a culture dish containing 25 μl Sage1-Step™ medium with serum protein supplement (Origio, Denmark) under oil and is then placed into the incubator (K Systems G210, Cooper Surgical, USA). Laser zona opening is performed on day 4 to facilitate passage of embryonic cfeDNA into the culture media. Each laser zona-opened embryo is transferred on day 4 to fresh 20 μl Sage1-Step medium with serum protein supplement (Origio, Denmark) and is cultured until blastocyst formation.

The following steps during culturing differ from Example 1.

In order to be able to eliminate maternal contamination, a further procedural step is conducted on day 1 (D1): careful removal of residual cumulus/corona cells is conducted by pipetting and washing with fresh medium at least 3 times with careful inspection under microscope. Further, on day 1 additional inspection and washing or stripping is performed as cells (CC) become looser after fertilization.

All fertilized oocytes are subsequently cultured individually from day 1 (D1) to day 4 (D4) in Sage 1-Step medium with serum protein supplement (Origio, Denmark) under oil, in 25-μL droplets.

Culturing of fertilized oocytes occurs in this example from D4 to day 5 or day 6 (D5/D6).

During culturing, laser zona breaching on D4 is conducted to allow extrusion of embryonic cfDNA into a culture media. After zona breaching, all embryos are washed three times in fresh media to remove residual cumulus/corona cells. Then each embryo is transferred in a separate fresh 15 μl drop of Global HP medium with HSA (LifeGlobal) under oil.

On Day 5/6, the expanding or expanded blastocysts with a visible inner cell muss are collapsed by a laser pulse to allow extrusion of blastocoel fluid containing cell free embryonic DNA into the culture media drop. After 5-10 min, the embryos were transferred into biopsy dish, and laser biopsy of the TE cells can follow under standard TE biopsy protocol.

### Sample Collection

Collection of spent embryo culture media and blastocoel fluid. The non-invasive and invasive preimplantation genetic testing (NIPGT-A and PGT-A) workflow is as described in Example 1. In brief, when blastocyst full expansion is observed, the blastocysts are collapsed by a single laser pulse at the junction of TE cells (infrared Zilos-tk™ or Lykoslaser™, Hamilton Thorne Biosciences, Beverley, MA) allowing release of blastocoel fluid (BF) into the media.

The spent embryo culture media (SEM) combined with blastocoel fluid (BF) are collected using 10 μl DNAse, RNAse & Pyrogen Free DIATEC Extended Filter Tips from each drop into empty DNAse and RNAse free 0.2 ml PCR tubes as one non-invasive PGT (NIPGT) sample.

After transferring the embryo to a biopsy dish, collection of the mixture of leaked BF together with embryo culture media (˜5 μL) as one miPGT sample is conducted using sterile single-use pipettes (to prevent contamination) in sterile RNase-DNase-free PCR tubes and is stored at −80 ° C. until analyzed. All collected samples (NIPGT and corresponding TE biopsy) were frozen. Control blank media samples (no embryo) are cultured under the same conditions to serve as negative controls.

Whole genome amplification, sequencing, and analysis. Similar to Example 1, whole genome amplification (WGA) is performed, according to manufacturer's instructions, using the SurePlex™ WGA (VeriSeq™ PGS Kit, Illumina). The WGA starts with enzymatic lysis of biopsied cells or NIPGT samples (5 μl SEM+BF) to release gDNA followed by pre-amplification and amplification steps using degenerative primers for uniform random whole genome amplification.

To improve the performance of library preparation of NIPGT samples, the WGA cell free DNA is enzymatically treated before it is used for library preparation. This involved Exo nuclease I treatment combined with Shrimp Alkaline phosphatase (Exo-SAP-I) to remove single stranded DNA. This step improves the yield of reads uniquely mapping to human genome and improves sequencing quality when samples are analyzed by next-generation sequencing (NGS).

The whole genome amplification (WGA) of DNA from NIPGT after treatment with Exo-SAP can be used also for PCR amplification followed by Sanger sequencing, Single base extension analysis or STR analysis.

A modified protocol for library prep is conducted using NexteraXT™ dual index set A-D (Illumina) that includes increase of amplification cycles from 12 to 16 and sequencing on NextSeq™ 550 to 0.5-1× genome coverage. This improves the informative rate of NIPGT samples. CNV analysis is performed with NxClinical™ software and analysis against a reference set from cell free embryonic DNA from euploid embryos.

WGA SurePlex protocol starts with direct pre-amplification of a 10 μl mi/ni-PGT (SEM+BF) sample. WGA products (SurePlex™ kit, Illumina) are quantified with the Qubit3.0-Fluorometer and their size distribution is assessed using 2100 BioAnalyzer (DNA high sensitivity chip, Agilent).

Samples are diluted to 0.2 ng/μl and a total of 1 ng from each sample and amplified using random primers. The kit contains 24 unique indexes added by amplification. Indexed DNA libraries are cleaned-up (AMPure XP beads 1:1 ratio) and normalized using magnetic beads. The normalized libraries are pooled, denatured, and sequenced using a MiSeq (single-end, 1×36 bp). Alignment and demultiplexing are done as part of the VeriSeq™ PGS protocol on MiSeq™ and CNV analysis and visualization were done using BlueFuse™ Multi (Illumina) software. Reporting was done using Hg39 reference with threshold for mosaicism of >30% and CNV changes >10 Mb.

For aneuploidy testing, SurePlex kit (BlueGnome) was used for whole genome amplification (WGA) of NIPGT and TE biopsy samples according to the manufactures instructions and quantified by Qubit 3.0 Fluorimeter (Thermo Fisher Scientific).

NGS by VeriSeq PGS (Illumina) is used to determine concordance rates for whole chromosome copy number (WCN) between NIPGT and corresponding TE biopsy samples.

To improve performance of library preparation of NIPGT samples the WGA using SurePlex kit with modification of pre-amplification stage according to manufacturer's protocol.

The protocol for preamplification involves preparing a pre-amplification cocktail of the following components: SurePlex pre-amp buffer (4.8 μl per sample) and SurePlex pre-amp enzyme (0.2 μl per sample). The pre-amplification cocktail components are combined and mixed well, for a total volume of 5 μl per sample. For each 10 μl of sample prepared, 5 μl of the pre-amplification cocktail is added, and the mixture is briefly centrifuged. Samples are incubated and thermocycled, with the number of cycles being increased (from manufacturer's protocol of 12) to 14.

WGA cell free DNA is concentrated 30 min/45 C and after enzymatically treating before it is used for library preparation. This involves Exo nuclease I treatment combined with Shrimp Alkaline phosphatase (Exo-SAP-IT) to remove single stranded DNA, thereby improving the yield of reads uniquely mapping to human genome and sequencing quality when samples are analyzed by NGS.

The WGA DNA from NIPGT after treatment with Exo-SAP can also be used for PCR amplification followed by Sanger sequencing, Single base extension analysis or short tandem repeat (STR) analysis.

A protocol for library prep using NexteraXT dual index set A-D (Illumina) involves amplification according to SurePlex summary protocol that includes an increase of amplification cycles (from manufacturer's protocol of 12) to 16 and sequencing on NextSeq 550 to 0.5-1× genome coverage. An amplification cocktail is prepared using SurePlex amplification buffer, SurePlex amplification enzyme, and Nuclease-free water (in amounts of 25 μl per sample; 0.8 μl per sample; and 34.2 μl per sample, respectively). For a volume of 60 μl of amplification cocktail per sample. The method improves the informative rate of NIPGT samples. CNV analysis is performed with NxClinical software and proprietary analysis against a reference set form cell free embryonic DNA from euploid embryos.

The DNA library so prepared is used to characterize the subject embryo.

Using fluorescently labelled short tandem repeat (STR) marker analysis of embryonic DNA (TE cells) and NIPGT samples, beneficial information is obtained on maternal contamination and identity of the samples.

### Examples Only

In the preceding description, for purposes of explanation, numerous details are set forth in order to provide a thorough understanding of the embodiments. However, it will be apparent to one skilled in the art that these specific details are not required.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

### REFERENCES

All publications, patents and patent applications mentioned in this specification are indicative of the level of skill those skilled in the art to which this invention pertains and are herein incorporated by reference to the same extent as if each individual publication, patent, or patent application was specifically and individually indicated to be incorporated by reference.

### Non-Patent Literature

Battaglia, R. et al. Identification of extracellular vesicles and characterization of miRNA expression profiles in human blastocoel fluid. Scientific reports 9, 84, doi:10.1038/s41598-018-36452-7 (2019).
Belandres, D., Shamonki, M. & Arrach, N. Current status of spent embryo media research for preimplantation genetic testing. Journal of assisted reproduction and genetics 36, 819-826, doi:10.1007/s10815-019-01437-6 (2019).
Ben-Nagi, J. et al. The First ongoing Pregnancy Following Comprehensive Aneuploidy Assessment Using a Combined Blastocenetesis, Cell Free DNA and Trophectoderm Biopsy Strategy. Journal of reproduction & infertility 20, 57-62 (2019).
Capalbo, A. et al. Diagnostic efficacy of blastocoel fluid and spent media as sources of DNA for preimplantation genetic testing in standard clinical conditions. Fertility and sterility 110, 870-879.e875, doi:10.1016/j.fertnstert.2018.05.031 (2018).
Dandouh, E. M., Balayla, J. & Garcia-Velasco, J. A. Impact of blastocyst biopsy and comprehensive chromosome screening technology on preimplantation genetic screening: a systematic review of randomized controlled trials. Reproductive biomedicine online 30, 281-289, doi:10.1016/j.rbmo.2014.11.015 (2015).
Darwish, E. & Magdi, Y. Artificial shrinkage of blastocoel using a laser pulse prior to vitrification improves clinical outcome. Journal of assisted reproduction and genetics 33, 467-471, doi:10.1007/s10815-016-0662-z (2016).
Dillon, L. W. et al. Production of Extrachromosomal MicroDNAs Is Linked to Mismatch Repair Pathways and Transcriptional Activity. Cell reports 11, 1749-1759, doi:10.1016/j.celrep.2015.05.020 (2015).
Fang, R. et al. Chromosome screening using culture medium of embryos fertilised in vitro: a pilot clinical study. Journal of translational medicine 17, 73, doi:10.1186/s12967-019-1827-1 (2019).
Friedenthal, J. et al. Next generation sequencing for preimplantation genetic screening improves pregnancy outcomes compared with array comparative genomic hybridization in single thawed euploid embryo transfer cycles. Fertility and sterility 109, 627-632, doi:10.1016/j.fertnstert.2017.12.017 (2018).
Galan, A. et al. Functional genomics of 5- to 8-cell stage human embryos by blastomere single-cell cDNA analysis. PloS one 5, e13615, doi:10.1371/journal.pone.0013615 (2010).
Fuchs Weizman, N. et al. Towards Improving Embryo Prioritization: Parallel Next Generation Sequencing of DNA and RNA from a Single Trophectoderm Biopsy. Scientific reports 9, 2853, doi:10.1038/s41598-019-39111-7 (2019).
Galluzzi, L. et al. Extracellular embryo genomic DNA and its potential for genotyping applications. Future science OA 1, Fso62, doi:10.4155/fso.15.62 (2015).
Guzman, L. et al. The number of biopsied trophectoderm cells may affect pregnancy outcomes. Journal of assisted reproduction and genetics 36, 145-151, doi:10.1007/s10815-018-1331-1 (2019).
Hammond, E. R., Shelling, A. N. & Cree, L. M. Nuclear and mitochondrial DNA in blastocoele fluid and embryo culture medium: evidence and potential clinical use. Human reproduction (Oxford, England) 31, 1653-1661, doi:10.1093/humrep/dew132 (2016).
Handyside, A. H. Noninvasive preimplantation genetic testing: dream or reality? Fertility and sterility 106, 1324-1325, doi:10.1016/j.fertnstert.2016.08.046 (2016).
He, H. et al. Neonatal outcomes of live births after blastocyst biopsy in preimplantation genetic testing cycles: a follow-up of 1,721 children. Fertility and sterility 112, 82-88, doi:10.1016/j.fertnstert.2019.03.006 (2019).
Heitmann, R. J., Hill, M. J., Richter, K. S., DeCherney, A. H. & Widra, E. A. The simplified SART embryo scoring system is highly correlated to implantation and live birth in single blastocyst transfers. Journal of assisted reproduction and genetics 30, 563-567, doi:10.1007/s10815-013-9932-1 (2013).
Ho, J. R. et al. Pushing the limits of detection: investigation of cell-free DNA for aneuploidy screening in embryos. Fertility and sterility 110, 467-475.e462, doi:10.1016/j.fertnstert.2018.03.036 (2018).
Huang, L. et al. Noninvasive preimplantation genetic testing for aneuploidy in spent medium may be more reliable than trophectoderm biopsy. Proceedings of the National Academy of Sciences of the United States of America 116, 14105-14112, doi:10.1073/pnas.1907472116 (2019).
Jiao, J. et al. Minimally invasive preimplantation genetic testing using blastocyst culture medium. Human reproduction (Oxford, England) 34, 1369-1379, doi:10.1093/humrep/dez075 (2019).
Kuznyetsov, V. et al. Evaluation of a novel non-invasive preimplantation genetic screening approach. PloS one 13, e0197262, doi:10.1371/journal.pone.0197262 (2018).
Kuznyetsov V, Madjunkova S, Abramov R, Antes R, Ibarrientos Z, Motamedi G, Zaman A, Kuznyetsova I, Librach C L. Minimally Invasive Cell-Free Human Embryo Aneuploidy Testing (miPGT-A) Utilizing Combined Spent Embryo Culture Medium and Blastocoel Fluid—Towards Development of a Clinical Assay. Sci Rep. 2020 Apr. 29;10 (1):7244. doi: PMID: 32350403.
Li, P. et al. Preimplantation Genetic Screening with Spent Culture Medium/Blastocoel Fluid for in Vitro Fertilization. Scientific reports 8, 9275, doi:10.1038/s41598-018-27367-4 (2018).
Madjunkova S et al. Non-invasive preimplantation genetic testing for monogenetic diseases and aneuploidies using cell free embryonic DNA, in: American Society of Human Genetics 68th Annual Meeting, San Diego, p 1270, Abstract 3007T (2018).
Maxwell, S. M. et al. Why do euploid embryos miscarry? A case-control study comparing the rate of aneuploidy within presumed euploid embryos that resulted in miscarriage or live birth using next-generation sequencing. Fertility and sterility 106, 1414-1419.e1415, doi:10.1016/j.fertnstert.2016.08.017 (2016).
Magli, M. C. et al. Preimplantation genetic testing: polar bodies, blastomeres, trophectoderm cells, or blastocoelic fluid? Fertility and sterility 105, 676-683.e675, doi:10.1016/j.fertnstert.2015.11.018 (2016).
Mukaida, T., Oka, C., Goto, T. & Takahashi, K. Artificial shrinkage of blastocoeles using either a micro-needle or a laser pulse prior to the cooling steps of vitrification improves survival rate and pregnancy outcome of vitrified human blastocysts. Human reproduction (Oxford, England) 21, 3246-3252, doi:10.1093/humrep/del285 (2006).
Munne, S. Status of preimplantation genetic testing and embryo selection. Reproductive biomedicine online 37, 393-396, doi:10.1016/j.rbmo.2018.08.001 (2018).
Neal, S. A. et al. High relative deoxyribonucleic acid content of trophectoderm biopsy adversely affects pregnancy outcomes. Fertility and sterility 107, 731-736.e731, doi:10.1016/j.fertnstert.2016.11.013 (2017).
Palini, S. et al. Genomic DNA in human blastocoele fluid. Reproductive biomedicine online 26, 603-610, doi:10.1016/j.rbmo.2013.02.012 (2013).
Poli, M. et al. Past, Present, and Future Strategies for Enhanced Assessment of Embryo's Genome and Reproductive Competence in Women of Advanced Reproductive Age. Frontiers in endocrinology 10, 154, doi:10.3389/fendo.2019.00154 (2019).
Popovic, M. et al. Chromosomal mosaicism in human blastocysts: the ultimate challenge of preimplantation genetic testing? Human reproduction (Oxford, England) 33, 1342-1354, doi:10.1093/humrep/dey106 (2018).
Rubio, C., Rodrigo, L., et al. Clinical application of embryo aneuploidy testing by next-generation sequencing. Biology of reproduction, 101 (6):1083-1090, doi:10.1093/biolre/ioz019 (2019).
Rubio, C., Rienzi, L., et al. Embryonic cell-free DNA versus trophectoderm biopsy for aneuploidy testing: concordance rate and clinical implications. Fertility and sterility 112, 510-519, doi:10.1016/j.fertnstert.2019.04.038 (2019).
Rule, K., Chosed, R. J., Arthur Chang, T., David Wininger, J. & Roudebush, W. E. Relationship between blastocoel cell-free DNA and day-5 blastocyst morphology. Journal of assisted reproduction and genetics 35, 1497-1501, doi:10.1007/s10815-018-1223-4 (2018).
Shamonki, M. I., Jin, H., Haimowitz, Z. & Liu, L. Proof of concept: preimplantation genetic screening without embryo biopsy through analysis of cell-free DNA in spent embryo culture media. Fertility and sterility 106, 1312-1318, doi:10.1016/j.fertnstert.2016.07.1112 (2016).
Stigliani, S., Anserini, P., Venturini, P. L. & Scaruffi, P. Mitochondrial DNA content in embryo culture medium is significantly associated with human embryo fragmentation. Human reproduction (Oxford, England) 28, 2652-2660, doi:10.1093/humrep/det314 (2013).
Sunkara, S. K., Antonisamy, B., Selliah, H. Y. & Kamath, M. S. Pre-term birth and low birth weight following preimplantation genetic diagnosis: analysis of 88 010 singleton live births following PGD and IVF cycles. Human reproduction (Oxford, England) 32, 432-438, doi:10.1093/humrep/dew317 (2017).
Tobler, K. J. et al. Blastocoel fluid from differentiated blastocysts harbors embryonic genomic material capable of a whole-genome deoxyribonucleic acid amplification and comprehensive chromosome microarray analysis. Fertility and sterility 104, 418-425, doi:10.1016/j.fertnstert.2015.04.028 (2015).
Vera-Rodriguez, M. et al. Origin and composition of cell-free DNA in spent medium from human embryo culture during preimplantation development. Human reproduction (Oxford, England) 33, 745-756, doi:10.1093/humrep/dey028 (2018).
Victor, A. R. et al. Assessment of aneuploidy concordance between clinical trophectoderm biopsy and blastocyst. Human reproduction (Oxford, England) 34, 181-192, doi:10.1093/humrep/dey327 (2019).
Vyas, P., Balakier, H. & Librach, C. L. Ultrastructural identification of CD9 positive extracellular vesicles released from human embryos and transported through the zona pellucida. Systems biology in reproductive medicine 65, 273-280, doi:10.1080/19396368.2019.1619858 (2019).
Wu, H. et al. Medium-based noninvasive preimplantation genetic diagnosis for human alpha-thalassemias-SEA. Medicine 94, e669, doi:10.1097/md.0000000000000669 (2015).
Xu, J. et al. Noninvasive chromosome screening of human embryos by genome sequencing of embryo culture medium for in vitro fertilization. Proceedings of the National Academy of Sciences of the United States of America 113, 11907-11912, doi:10.1073/pnas.1613294113 (2016).
Zeng, M., Su, S. & Li, L. The effect of laser-assisted hatching on pregnancy outcomes of cryopreserved-thawed embryo transfer: a meta-analysis of randomized controlled trials. Lasers in medical science 33, 655-666, doi:10.1007/s10103-017-2372-x (2018).
Zhang, S. et al. Number of biopsied trophectoderm cells is likely to affect the implantation potential of blastocysts with poor trophectoderm quality. Fertility and sterility 105, 1222-1227.e1224, doi:10.1016/j.fertnstert.2016.01.011 (2016).
Zhang, Y. et al. Molecular analysis of DNA in blastocoele fluid using next-generation sequencing. Journal of assisted reproduction and genetics 33, 637-645, doi:10.1007/s10815-016-0667-7 (2016).

1. A non-invasive method for genetic screening prior to implantation of an embryo, said method comprising:
culturing fertilized oocytes in a culture medium on day 1 of fertilization;
removal of residual cumulus/corona cells from culture medium by pipetting and washing with fresh medium on culture day 1 to reduce maternal contamination;
isolating fertilized oocytes and culturing individually from day 1 to day 4 in a culture medium comprising a serum protein supplement;
conducting laser zona breaching on day 4 to allow embryonic cell free DNA (cfDNA) into said culture medium, and subsequently washing the fertilized oocytes with fresh medium to produce a medium containing day 4 cfDNA;
transferring said washed fertilized oocytes on day 4 into fresh culture medium under oil, and culturing until day 5, day 6, or day 7 to thereby form an expanded blastocyst;
transferring said expanded blastocyst on day 5, day 6, or day 7 (day 5/6/7) into a fresh drop of culture medium;
exposing said expanded blastocyst to a laser pulse to extrude blastocoel fluid containing embryonic cell free DNA (cfDNA) in the fresh drop of culture medium to obtain day 5/6/7 cfDNA; and
conducting genetic screening of the cfDNA using whole genome amplification (WGA) prior to implantation of the embryo.

2. The method for genetic screening according to claim 1, wherein the step of conducting genetic screening comprises aneuploidy testing using said WGA to determine whole chromosome copy number (WCN) as an indicator of aneuploidy.

3. The method of claim 1, wherein the removal of residual cumulus/corona cells from culture medium by pipetting and washing comprises at least three washes with fresh culture medium and microscopic inspection.

4. The method of claim 1, wherein the culture medium for said individually cultured fertilized oocytes from day 1 to day 4 comprises Sage 1-Step medium, under oil in a culture medium droplet of about 25 μL.

5. The method of claim 1, wherein the washing after the laser zona breaching on day 4 comprises three washings to remove residual cumulus/corona cells.

6. The method of claim 5, wherein following said three washings to remove the residual cumulus/corona cells, the fertilized oocyte is transferred to a fresh culture medium comprising Global HP medium with human serum albumen (HAS), under oil, in a culture medium droplet of about 15 μl.

7. The method of claim 1, wherein said expanded blastocyst on day 5/6/7 comprises a visible inner cell muss prior to said laser pulse.

8. The method of claim 1, wherein the genetic screening of the cfDNA is assessed in the spent culture media, in the blastocoel fluid, or in both.

9. The method of claim 2, wherein cfDNA is enzymatically treated with Exo nuclease I and Shrimp Alkaline phosphatase (Exo-SAP-IT) to remove single stranded DNA prior to whole genome amplification (WGA).

10. The method of claim 2, wherein whole genome amplification (WGA) is conducted using a SurePlex™ kit, quantified with a Qubit 3.0™ fluorimeter, and next-generation sequencing (NGS) is conducted with VeriSeq™ PGS.

11. The method of claim 10, wherein the whole genome amplification (WGA) with the SurePlex™ kit employs 14 pre-amplification cycles for preparation of a library of sequences.

12. The method of claim 10, wherein DNA resulting from WGA is subjected to PCR amplification followed by: Sanger sequencing, Single base extension analysis, or short tandem repeat (STR) analysis.

13. The method of claim 12, wherein fluorescent markers are used for short tandem repeat (STR) analysis.

14. The method of claim 1, wherein the step of conducting genetic screening of the cfDNA using whole genome amplification (WGA) employs preparation of a cfDNA library using NexteraXT™ dual index set A-D with 16 amplification cycles.

15. The method of claim 2, wherein the step of conducting genetic screening comprises copy number variation (CNV) analysis with NxClinical™ software against a reference set from cell free embryonic DNA from euploid embryos.

16. The method of claim 1, further comprising the step of implantation of the embryo if it satisfies a requirement of the genetic screening, into a human subject.

17. The method of claim 16, further comprising the step of freezing the embryo that satisfies the requirement of the genetic screening, prior to implanting in the human subject.

18. The method of claim 2, further comprising the step of implantation of the embryo if aneuploidy is not indicated in the genetic screening, into a human subject.

19. The method of claim 16, further comprising the step of freezing the embryo if aneuploidy is not indicated, prior to implanting in the human subject.

20. (canceled)
